(19) United States
(12) Patent Application Publication
(10) Pub. No.: US 2010/0159582 A1
(43) Pub. Date: Jun. 24, 2010

(54) DISPOSABLE MULTIPLEX POLYMERASE CHAIN REACTION (PCR) CHIP AND DEVICE

(75) Inventors: Asma ISMAIL, Penang (MY); Sugumar DHARMALINGAM, Penang (MY); Lingxue KONG, Penang (MY)

Correspondence Address: Workman Nydegger;1000 Eagle Gate Tower, 60 East South Temple, Salt Lake City, UT, 84111, US

(73) Assignee: UNIVERSITI SAINS MALAYSIA, Penang (MY)

(21) Appl. No.: 12/510,056

(22) Filed: Jul. 27, 2009

Related U.S. Application Data
(63) Continuation of application No. PCT/MY2008/000190, filed on Dec. 18, 2008.

(51) Int. Cl. C12M 1/00

(52) U.S. Cl. 435/303.1

(57) ABSTRACT

A polymerase chain reaction (PCR) device including a chip assembly, a plurality of chambers being provided in said chip assembly adapted to hold samples, heating means wherein said chip assembly being located on said heating means whereby said chip assembly is allowed to operatively rotate on said heating means, a rotary wheel aiding said chip rotation and wherein said heating means comprises of plural temperature zones in a manner that on rotation of said chip means said sample chamber is shifted from one temperature zone to another by means of a rotary-linear motion system.

### FIELD OF INVENTION

The present invention relates to multiplex Polymerase Chain Reaction (PCR) device. More particularly, the invention relates to a disposable PCR device comprising sample chambers such that the said chambers have the proviso of shifting from one temperature zone to another by means of rotary-linear motion system.

### BACKGROUND OF INVENTION

Polymerase Chain Reaction (PCR) is an important method that can amplify specific Deoxyribonucleic acid (DNA) in an exponential value (as large as 2³⁰ times), without simultaneous amplification of other genetic material present in the solution. This technology is a major breakthrough for molecular biology applications, which was first introduced in the year 1986. The amplification method mimics the natural process of replication and repair of DNA and expression of proteins which regularly occur within natural biochemical processes.

The replication of the DNA from a single strand of DNA is performed by specific enzymes, such as DNA polymerase. With the manipulation of temperature for denaturation and hybridization of the double stranded DNA, large copies of a specific DNA can be produced.

To copy a DNA, polymerase requires two other components. First, an ample supply of the four nucleotide bases, which are building blocks of every piece of DNA. They are represented by the letters A, C, G and T, which stands for adenine, cytosine, guanine, and thymine, respectively. The A on a strand always pairs with the T on the other strand, C always pairs with G. These two strands are said to be complementary to each other. The second component is the primers. They are short synthetic chains of complementary nucleotides to the genetic sequence on either flank of the targeted section in the DNA strand. DNA polymerase cannot copy a chain of DNA without the primers. The primers hybridize on either ends of the targeted section, and the polymerase enzyme constructs the rest of the chain between them, from the raw materials (single nucleotides).

The copying of a single DNA strands goes through 3 major steps, which is known as the PCR. The PCR mixture contains the target DNA, primers and nucleotides and DNA polymerase. The first step, known as denaturing, separates the two DNA strands in the double helix. This is done by simply heating the DNA at 90°-95° centigrade for about 30 seconds. However, at this temperature, the primers cannot bind to the separated DNA strands. Therefore, the mixture is cooled to a lower temperature of 55°-64° degrees centigrade, depending on the DNA. At this temperature, the primers bind or anneal to the ends of the DNA strands, which takes about 20 seconds. The final step is completing the copying of the DNA. Since the DNA polymerase works best at around 75° centigrade, the temperature of the mixture is increased. At this temperature, the DNA polymerase begins building or adding up the single nucleotides to the primers and eventually makes a complimentary copy of the template (know as extension). This completes the PCR cycle. At the end of this cycle, each piece of DNA in the mixture has been duplicated. When the cycle is repeated 30 or more times, more than 1 billion copies of a single DNA can be produced. The cycle of denaturation, annealing and extension is done through thermal cycling, which contributes to the idea of miniaturization of this process.

After the discovery of micro technology in the early 1950s for realizing integrated semiconductor structures for microelectronic chips, these lithography-based technologies were soon applied in pressure sensor manufacturing as well in the mid 1960s. Next to pressure sensors, airbag sensors and other mechanically movable structures, fluid handling devices were developed. The first Lab on Chip (LoC) analysis system was a gas chromatograph, developed in 1975 by S. C. Terry at Stanford University. However, only at the end of the 1980's, and beginning of the 1990's, the LoC research started to seriously grow as a few research groups in Europe developed micropumps, flow-sensors and the concepts for integrated fluid treatments for analysis systems. These micro total chemical analysis system (μTAS) concepts demonstrated that integration of pre-treatment steps, usually done at lab-scale, could extend the simple sensor functionality towards a complete laboratory analysis, including e.g. additional cleaning and separation steps. A big boost in research and commercial interest came in the mid 1990's, when μTAS technologies turned out to provide interesting tooling for genomics applications, like capillary electrophoresis and DNA microarrays.

The added value was not only limited to integration of lab processes for analysis but also the characteristic possibilities of individual components and the application to other, non-analysis, lab processes. Although the application of LOCs is still novel and modest, a growing interest of companies and applied research groups is observed in different fields such as analysis (e.g. chemical analysis, environmental monitoring, medical diagnostics and cellomics) but also in synthetic chemistry (e.g. rapid screening and microreactors for pharmaceutics). Besides further application developments, research in LoC systems is expected to extend towards downscaling of fluid handling structures as well, by using nanotechnology.

LoCs may provide advantages, very specifically for their applications. Typical advantages are:
low fluid volumes consumption, because of the low internal chip volumes, which is beneficial for e.g. environmental pollution (less waste), lower costs of expensive reagents and less sample fluid is used for diagnostics
higher analysis and control speed of the chip and better efficiency due to short mixing times (short diffusion distances), fast heating (short distances, high wall surface to fluid volume ratios, small heat capacities)
better process control because of a faster response of the system (e.g. thermal control for exothermic chemical reactions)
compactness of the systems, due to large integration of functionality and small volumes
massive parallelization due to compactness, which allows high-throughput analysis
lower fabrication costs, allowing cost-effective disposable chips, fabricated in mass production
safer platform for chemical, radioactive or biological studies because of large integration of functionality and low stored fluid volumes and energies

Conventional macro scale PCR devices typically consists of computer thermocyclers and reaction vials, containing the PCR mixture. Conventional PCR devices usually achieve temperature ramping rate of about 1-2 degrees C. per second in the temperature range relevant for PCR. The PCR process for 20-35 cycles can be completed typically in 30 to 180 minutes, depending on the capability of the thermocyclers. The reason for the lower ramping is due to the high thermal capacity of the material of the PCR reaction system. The PCR products can be analyzed using traditional slab-gel electrophoresis.

With the advancement in microfabrication, the first PCR chip was introduced by Northrup et.al. From thereon, many types of PCR chips technology have been introduced. The basis of PCR chips are faster DNA amplification rates as the result of smaller thermal capacity and larger heat transfer rate between the PCR mixture and temperature controlled components. This is accomplished by using small size, fast temperature ramping rates, low cost, lower consumption of samples, and high integration.

However, with the miniaturization, the effects related to non-specific adsorption of biological samples to the surfaces of the channel and viscoelastic flow behaviour may become significant as a result of the increased surface to volume ratio which may inhibit PCR amplification in microfluidic devices.

Any discussion of the prior art throughout the specification should in no way be considered as an admission that such prior art is widely known or forms part of common general knowledge in the field.

### SUMMARY OF INVENTION

Accordingly there is provided a polymerase chain reaction (PCR) device including a chip assembly, a plurality of chambers being provided in said chip assembly adapted to hold samples, heating means wherein said chip assembly being located on said heating means whereby said chip assembly is allowed to operatively rotate on said heating means, a rotary wheel aiding said chip rotation and wherein said heating means comprises of plural temperature zones in a manner that on rotation of said chip means said sample chamber is shifted from one temperature zone to another by means of a rotary-linear motion system.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The present invention consists of several novel features and a combination of parts hereinafter fully described and illustrated in the accompanying description and drawings, it being understood that various changes in the details may be made without departing from the scope of the invention or sacrificing any of the advantages of the present invention.

### BRIEF DESCRIPTION OF THE DRAWINGS

The present invention will be fully understood from the detailed description given herein below and the accompanying drawings which are given by way of illustration only, and thus are not limitative of the present invention, wherein:

FIG. 1 illustrates the PCR chips assembled to the PCRDisc wheel.

FIG. 2 illustrates the disposable polymer PCR chips with four sample chambers.

FIG. 3 illustrates the heater assembly of the PCRDisc.

FIG. 4 represents the schematic diagram of the assembled PCRDisc rotary wheel.

FIG. 5 illustrates the assembled PCRDisc device.

### DETAILED DESCRIPTION OF THE PREFERRED EMBODIMENTS

The present invention relates to a Polymerase Chain Reaction Disc (PCRDisc) utilizing the advantages of the stationary chamber and continuous flow PCR device. Hereinafter, this specification will describe the present invention according to the preferred embodiments of the present invention. However, it is to be understood that limiting the description to the preferred embodiments of the invention is merely to facilitate discussion of the present invention and it is envisioned that those skilled in the art may devise various modifications and equivalents without departing from the scope of the appended claims.

The following detailed description of the preferred embodiments will now be described in accordance with the attached drawings, either individually or in combination.

The invention relates to a disposable PCR device comprising sample chambers such that the said chambers have the proviso of shifting from one temperature zone to another by means of rotary-linear motion system. Instead of using an external pump to move the sample to different temperature zone, the said device shifts the sample chamber from one temperature zone to another by using the rotary-linear motion system. Each individual sample chamber temperatures are controlled individually. In this way, several different Deoxyribonucleic acid (DNA) samples with different annealing temperatures can be amplified simultaneously in a single process.

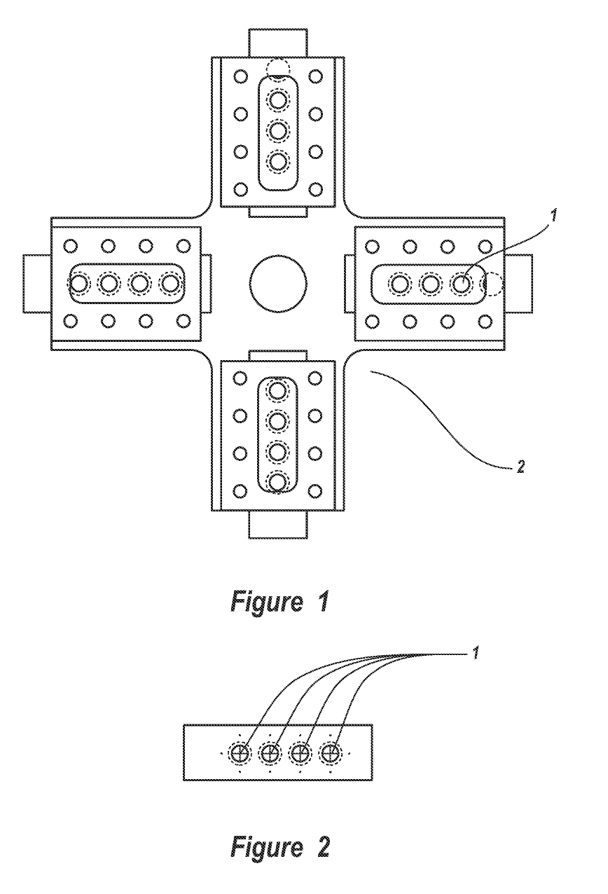

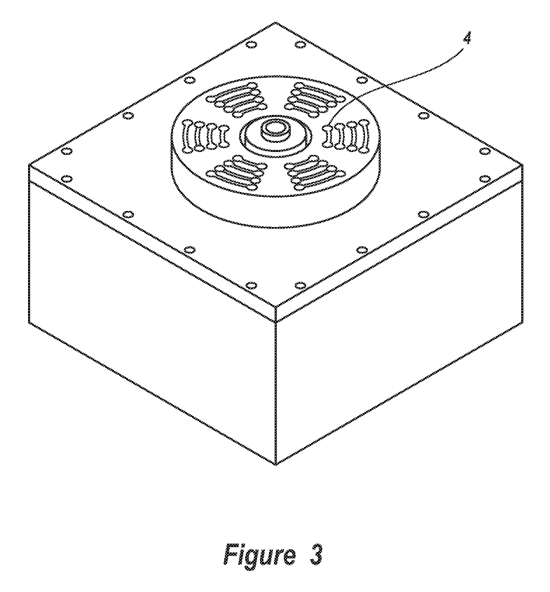

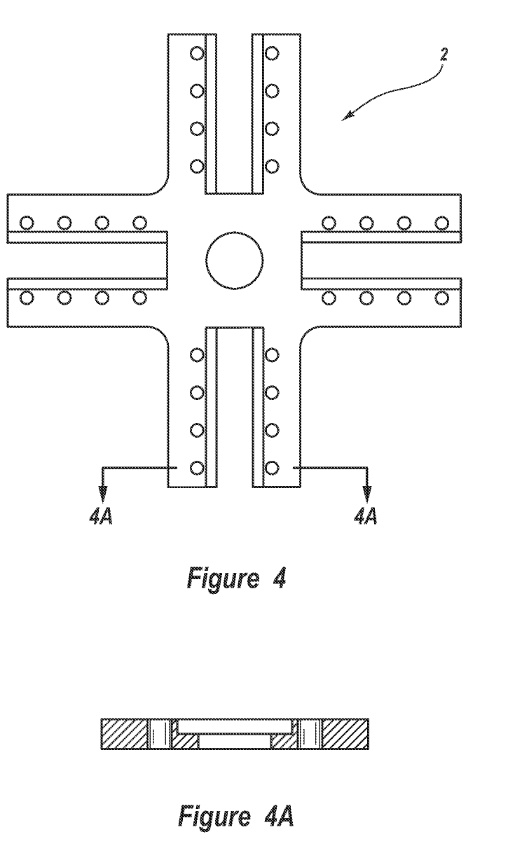

In this particular embodiment of the inventive concept, the PRCDisc has 16 sample chambers 1. The number of individually controlled heaters is also 16 units. FIG. 1 shows the illustration of the PCRDisc wheel 2. The sample chambers 1 are made of individual cartridges 3 that are made of polymer material to reduce the cost of fabrication. Each cartridge 3 has a total of four sample chambers 1 as shown in FIG. 2. Special housing is designed and fabricated to accommodate the heaters and mount the PCRDisc wheel (FIGS. 3 and 4). Additionally, a separate system of motor control unit is developed to accommodate the rotational and linear movement of the disc.

As disclosed, the disc 2 can have up to 16 chambers 1. However, for the proposed system, only 12 chambers are being utilized for the experiments. This is due to the limitation on the number of heaters available and the number of physical channels available for the National Instruments control system. In this system, the layout of the heaters 4 is as shown in FIG. 3. There are 3 heaters for each of the denaturing and annealing temperature zones/rows and 2 rows of 3 heaters each for the extension temperature zone. The reason for the additional row of heaters for the extension temperature zone is to minimize the total cycle time. As explained earlier, extension time depends on the base pair length of the template DNA. Denaturing and annealing duration is minimal. Since the denaturing process occurs once the required temperature is achieved, therefore it does not need additional dwelling time. And as for the annealing process, due to the short strands of the primers, this process completes within a short period of time. In order for the extension process to complete the polymerase chain reaction, it is decided to double the duration required of that of denaturing and annealing process. This is done by having 2 rows of extension heaters next to each other after the annealing temperature row. For short base pair DNA amplification (less than 100 base pairs), the number of extension temperature rows can be reduced to one only. In this case, the system can be reconfigured to have only three temperature zones instead of four.

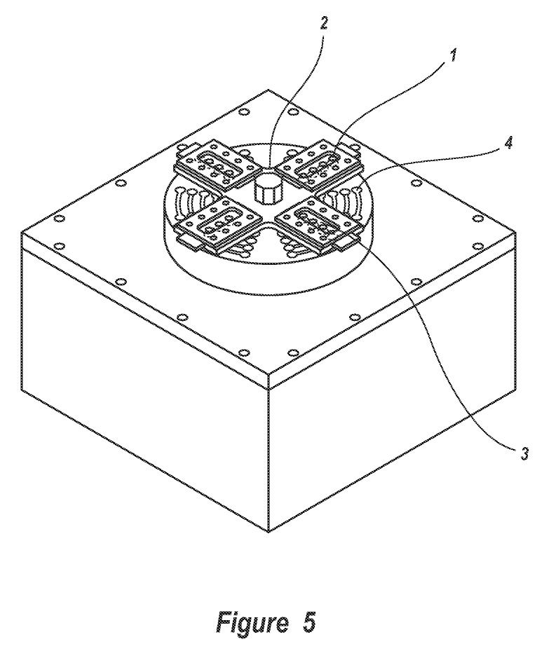

The sample chambers 1 are rotated in a clock wise direction using the rotary system to move it from one temperature zone to another (see FIG. 5). Once the sample chambers are positioned on top of the heaters 4, the whole disc 2 is retracted downward to press on to the heaters 4 by using the linear motion control system. In order for all the sample 1 chambers to come in perfect contact with the heaters 4, each heater is loaded with a spring for it to retract a few millimeters from its original position when pressed with some force. Once the disc 2 is in lower position (pressed against the heaters), the disc 2 will be allowed to remain in this position for it to complete the PCR process for a pre-determined duration (depending on the PCR sample). Once the duration is over, the disc 2 is pushed upward using the using the linear motion system and then the disc is rotated 90° to the next row of heaters 4. Thereafter, the same linear movement is executed. The sample will complete one complete PCR cycle after the sample chambers are rotated 3600 from the initial heating at the denaturing row. By controlling the number of rotary motion of the disc, the number of PCR cycles can be set. Therefore, a total of 12 samples can be amplified simultaneously within a short duration. Since the heater temperatures are controlled individually, the 3 or 4 annealing temperatures can be set for annealing row heaters. With this method, 3 or 4 different PCR samples with different annealing temperatures can be amplified in one disc. This method can be aptly named as “PCR chip multiplexing”.

1. A polymerase chain reaction (PCR) device including:
i. a chip assembly;
ii. a plurality of chambers being provided in said chip assembly adapted to hold samples;
iii. a heating means, wherein said chip assembly being located on said heating means whereby said chip assembly is allowed to operatively rotate on said heating means; and
iv. a rotary wheel aiding said chip rotation,
wherein said heating means comprises of plural temperature zones in a manner that on rotation of said chip means said sample chamber is shifted from one temperature zone to another by means of a rotary-linear motion system.

2. The device as claimed in claim 1 wherein said chip includes individual cartridges.

3. The device as claimed in claim 2 wherein said cartridge includes at least four sample chambers.

4. The device as claimed in claim 1 wherein said device includes a special housing to accommodate said heating means so as to mount the rotary wheel.

5. The device as claimed in claim 1, additionally including a motor control unit adapted to provide linear and rotational motion to the chip assembly.

6. The device as claimed in claim 5 wherein said motor control unit includes a linear motion control means adapted to retract the chip assembly downward to locate on the appropriate temperature zone of heating means.
